(19) United States
(12) Patent Application Publication
(10) Pub. No.: US 2003/0078172 A1
(43) Pub. Date: Apr. 24, 2003

(54) Foaming cosmetic cream

(75) Inventors: Carole Guiramand, Jouy en Josas (FR); Valerie Hurel, Gif/s/Yvette (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/245,569

(22) Filed: Sep. 18, 2002

(30) Foreign Application Priority Data
Sep. 20, 2001 (FR) 01 12151

(51) Int. Cl. A61K 7/50; A61K 7/48

(52) U.S. Cl. 510/135; 510/159; 510/424; 510/428

(57) ABSTRACT

The present application relates to a foaming composition for topical application containing at least one wax and a surfactant system such that at least one direct hexagonal or cubic paracrystalline phase appears when the temperature increases above 30° C. and such that this paracrystalline phase remains present up to at least 45° C.

The present invention relates to a rinsable foaming composition preferably in a form constituting a cream for topical application, which comprises a wax and a specific surfactant system and which exhibits good physical stability up to at least 45° C., and to its preparation and use in the cosmetic or dermatological fields, in particular as products for cleaning or removing make-up from the skin, scalp and/or hair.

### BACKGROUND OF THE INVENTION

Cleansing the skin is very important for caring for the face. It must be as efficient as possible because greasy residues, such as excess sebum, the remnants of cosmetic products used daily, and make-up products, in particular waterproof products, accumulate in the skin folds and can block the pores of the skin and result in the appearance of spots.

Several main types of skin cleansing products are known: foaming detergent aqueous lotions and gels, rinsable cleansing anhydrous oils and gels, and foaming creams.

Rinsable anhydrous oils and gels have a cleansing action by virtue of oils present in these formulations. These oils make it possible to dissolve fatty residues and to disperse make-up pigments. These products are effective and well tolerated. They exhibit the disadvantage of being heavy, of not foaming and of not conferring a feeling of freshness on application, which is disadvantageous from a cosmetic viewpoint.

Furthermore, foaming detergent aqueous lotions and gels have a cleansing action by virtue of the surfactants, which suspend the fatty residues and the pigments of the make-up products. They are effective and pleasant to use because they foam and because they are easy to remove. However, the lotions are generally fairly fluid, which makes them sometimes tricky to handle, and it is difficult to thicken the gels while retaining good foaming properties.

In order to obtain good foaming performances while having a thick composition, attempts have been made to prepare foaming creams. However, foaming creams exhibit the disadvantage of often being unstable towards heat and have been difficult to thicken without losing stability and foaming performance.

The term “foaming creams” is understood here to mean opaque and viscous compositions generally composed of an aqueous medium comprising a mixture of surfactants, such as fatty acid salts (soaps) or anionic, nonionic or amphoteric synthetic surfactants, and of other additives, such as, for example, polymers, polyols or fillers.

These creams, intended in particular for cleansing the skin, develop the foam when they are mixed with water. They can be used in two ways:

the first use consists in spreading the cream over the hands, in applying it to the face or to the body, and then massaging it in the presence of water to develop the foam directly on the face or the body;

the other possible use of this type of product consists in developing the foam in the palms of the hands before being applied to the face or the body.

In both cases, the foam is subsequently rinsed off.

The majority of foaming creams currently available commercially are unstable above 40° C. This means that, if they are stored for a few days at this temperature, they exhibit macroscopic phase separation, resulting in separation into at least two phases. Creams, thus phase-separated at a temperature markedly higher than ambient temperature, could be heterogeneous after returning to ambient temperature and thus are unusable because of the deterioration in the texture and in the foaming properties. The term “ambient temperature” is understood here to mean a moderate temperature, that is to say approximately (±10%) 20 to 25° C.

In point of fact, it is essential for this type of product to be stable over a wide temperature range. This is because, during its life, the product can be exposed to temperatures ranging from −20° C. to +45° C. minimum, depending upon the climatic, storage and/or transportation conditions. For example, it is necessary for a cream transported in a car which is subject to the risk of remaining exposed to the sun for a long period of time, that is to say at a temperature which can easily reach 50° C., to retain its stability. It is also necessary for these foaming creams to be able to be used in hot countries without their transportation and their storage presenting a problem.

It is well known that it is possible to prevent this phase separation of a foaming cream by increasing, by addition of polymers or of fillers, the consistency of the product subjected to temperatures of +40° to +45° C. However, in this case, the product becomes very stiff at moderate ambient temperature and no longer corresponds to the properties desired for application to the skin; in particular, it becomes difficult to mix it with water and to make it foam.

Furthermore, the preparation of more viscous creams is problematic in processing terms (homogenization by turbine difficult, risk of introduction of air, and the like) and poses the problem of a deterioration in the quality of mixing with water during use and the problem of starting to form a foam, which is difficult.

The need thus remains for a foaming cream which is stable up to at least 45° C., the cream appearance of which is maintained at ambient temperature even after changing to a higher temperature and which has a sufficient viscosity to constitute a cream while retaining good stability and the properties required for good cleaning, in particular good qualities of mixing with water and of foam formation.

### SUMMARY OF THE INVENTION

The inventors have discovered, surprisingly, that it is possible to achieve the aim of the invention and to obtain a foaming composition which is provided in the form of a cream and which has good stability, even at temperatures from +40 to +45° C., by incorporating a wax and by using a surfactant system such that at least one paracrystalline phase of direct hexagonal or cubic type appears when the composition is heated to a temperature of greater than 30° C. and such that this paracrystalline phase remains present up to at least 45° C.

The combination of wax and of the specific surfactant system in the composition of the invention makes it possible to obtain a cream which is simultaneously very stable and thick and creamy and which gives a foam having good cosmetic qualities, a good foaming performance and good spreading over the skin.

To obtain very good stability, it is preferable for the paracrystalline phase formed (or liquid crystal) to be of the direct hexagonal phase type. It is not necessary for the paracrystalline phase to be present at ambient temperature but it absolutely has to appear at least somewhere between greater than 30° C. and 45° C.

Foaming creams which do not exhibit a phase organization as mentioned above do not generally have satisfactory stability at 45° C. At this temperature, they undergo macroscopic phase separation between at least two phases and they are subsequently unsuitable for the desired use when they are again at ambient temperature.

Thus, a subject of the present application is a foaming composition for topical application comprising, in an aqueous medium, at least one wax and a surfactant system such that at least one paracrystalline phase of direct hexagonal and/or cubic type appears when the temperature increases above 30° C. and such that this paracrystalline phase remains present up to at least 45° C.

According to a preferred embodiment of the invention, at least one of the stages of preparation of the cream according to the invention is carried out in a screw mixer-extruder, in particular for the purpose of introducing larger amounts of wax, for example of greater than 2% by weight with respect to the total weight of the composition.

The composition of the invention is preferably provided in the form of a cream, that is to say a soft product, in contrast to a solid product, such as a stick. The cream preferably has a viscosity at approximately 25° C. generally ranging from approximately 20 to 250 poises, i.e. 2 to 25 Pa·s, more preferably from approximately 50 to 240 poises, i.e. 5 to 24 Pa·s, and better still 50 to 200 poises, i.e. 5 to 20 Pa·s, this viscosity being measured with a Rheomat 180.

The composition obtained, although comprising a wax, gives a foam which spreads homogeneously and which is of good quality (fine bubbles, good foam density, softness and ease of rinsing).

The paracrystalline phase or phases present above +30° C. can be of direct hexagonal or cubic type or can be a mixture of these two phases or a mixture of one of these phases or of both these phases with a phase of lamellar type. The paracrystalline phase(s) preferably comprise(s) at least one direct hexagonal phase.

The terms “lamellar phase”, “direct hexagonal phase” and “cubic phase” are given, in the present application, the meanings which a person skilled in the art generally gives to them.

Thus, the term “lamellar phase” (phase D according to Ekwall, see Advances in Liquid Crystals, vol. 1, page 1-143, Acad. Press, 1975, edited by G. H. Brown) is understood to mean a liquid crystal phase with plane symmetry comprising several amphiphilic bilayers arranged in parallel and separated by a liquid medium which is generally water.

The term “direct hexagonal phase” (phase F according to Ekwall, see Advances in Liquid Crystals, vol. 1, page 1-143, Acad. Press, 1975, edited by G. H. Brown) is understood to mean a liquid crystal phase corresponding to a hexagonal arrangement of parallel cylinders composed of an amphiphil and separated by a liquid medium which is generally water. In a direct hexagonal phase, the continuous medium is aqueous.

The term “cubic phase” is understood to mean a phase organized in a bipolar manner into separate hydrophilic and lipophilic domains, in close contact which form a thermodynamically stable three-dimensional network with cubic symmetry. Such an organization has been described in particular in “La Recherche”, Vol. 23, pp. 306-315, March 1992, and in “Lipid Technology”, Vol. 2, No. 2, pp. 42-45, April 1990. Depending upon the arrangement of the hydrophilic and lipophilic domains, the cubic phase is said to be of normal or inverted type. The term “cubic phase” used according to the present invention includes, of course, various types of cubic phases.

A more precise description of these phases can be found in Revue Française des Corps Gras, No. 2, February 1969, pp.87 to 111 (Lachampt and Vila, “Textures des phases paracristallines” [Textures of paracrystalline phases]).

Various techniques can be used to identify the constituent phases of the cream and in particular (1) small-angle and large-angle X-ray diffraction measurements and (2) observation by optical microscopy in polarized light.

X-ray Diffraction Technique

The X-ray diffraction technique is known as being one of the most relevant for demonstrating the organizations of paracrystalline phases, in particular within a sample. X-ray diffraction measurements can be carried out using a CGR Sigma 2060 generator equipped with an Inel tube comprising a Cu anticathode and a linear focusing chamber installed in symmetrical transmission. The samples are introduced at ambient temperature into a measurement cell closed off by Mylar or Capton windows and placed in a thermally regulated sample holder.

The diffraction spectra obtained with a wavelength λ=1.54 angstroms (Kα line of copper) are recorded using a photostimulable phosphor screen scanned by a Molecular Dynamics Phosphorlmager PSI laser scanning module. The detector/sample distance is adjusted to 133 mm, which gives access to lattice distances of between approximately 3 and 110 angstroms. The spectra are recorded at various set temperatures.

With this technique, the paracrystalline phases are characterized by the presence, at small diffraction angles, of a series of several fine lines due to Bragg reflections which correspond to distances: d1, d2. . . dn with distance ratios d1/d1, d1/d2, . . . , d1/dn which are characteristic of each type of phase, as indicated, for example, in “La structure des colloïdes d'association I. Les phases liquides cristallines des systemes amphiphile-eau” [The structure of association colloids, I. The crystalline liquid phases of amphiphile-water systems], V. Luzzati, H. Mustachi, A. Skoulios and F. Husson, Acta Cryst. (1960), 13, 660-667 or in Biochimica et Biophysica Acta (1990), 1031, pp. 1 to 69, by J. M. Seddon. Thus, for a phase with a lamellar structure and in particular for the paracrystalline phase of fluid lamellar type generally denoted by Lα and also known as neat phase, the distance ratios are equal to: 1, 2, 3, 4, . . . For the paracrystalline phase of direct hexagonal type generally denoted by H1 or E and also known as middle phase, the distance ratios are equal to: 1, √3, 2, √7, . . . . At large diffraction angles, the paracrystalline phases exhibit a band centred over a distance of the order of 4.5 angstroms, whereas the crystalline phases result in fine lines.

Observations by Optical Microscopy

Observations by optical microscopy in polarized light also contribute to the identification of paracrystalline phases, in particular when the number of lines observed by X-ray diffraction is insufficient to unambiguously establish the nature of the paracrystalline phases present.

Optical microscopy observations in polarized light are carried out, for example, using a Laborlux S (Leitz) microscope equipped with an objective with a magnification of 10, with a system of cross polarizers and with a heating stage (Mettler FP80/FP82). The sample is deposited between microscope slide and coverglass and covered with a second slide and assembly is sealed via a Parafilm® seal. The observations are made at various set temperatures or by temperature scanning at 2° C. min between ambient temperature and approximately 95° C.

It is known, for example, that isotropic micellar solutions are non-birefringent, that paracristalline phases of cubic type are also non-birefringent and that paracrystalline phases of direct or inverted hexagonal fluid lamellar type exhibit, in polarized light, various characteristic textures described, for example, in “Textures des phases paracrystallines rencontrées dans les diagrammes d'équilibre: agents de surface, lipides, eau” [Textures of paracrystalline phases encountered in equilibrium diagrams: surfactants, lipids, water], F. Lachampt and R. M. Vila, Revue Française des corps gras (1969), 2, 87-111 or in “The aqueous phase behavior of surfactants”, Robert G. Laughlin, Academic Press, (1996), pp. 521-546.

Viscosity Characteristics

The compositions according to the invention preferably constitute more or less fluid creams having, as indicated above, a viscosity at 25° C. generally ranging from approximately 2 to 25 Pa·s, preferably from approximately 5 to 24 Pa·s and better still 5 to 20 Pa·s (viscosity measured with a Rheomat 180). These creams are characterized by values of the moduli |G^{*}|, G′ and G″, at a temperature of 25° C., ranging from 10² to 10⁶ Pa.

G^{*}, G′ and G″ are viscoelastic parameters used to measure the physical properties of viscoelastic fluids, as explained in “An introduction to rheology” by H. A. Barnes, J. F. Hutton and K. Walters, pages 46 to 54 (published by Elsevier-1989).

G^{*} is the complex modulus and G′ and G″ are the components of G^{*}:G^{*}=G′+iG″. G′ and G″ are respectively the storage modulus and the loss modulus and i is equal to (−1)^{½}. The components G′ and G″ of the complex modulus are obtained from the relationship between the oscillatory stress and the oscillatory strain.

The rheological measurements of |G^{*}|, G′ and G″ are generally made using a Haake RS150 rheometer at a temperature of 25° C. with measuring bodies possessing cone-plate geometry, the diameter of the cone and the size of the plate being 20 mm and the angle of the cone being 1° and the gap between the cone and the plate being 0.05 mm.

To make dynamic measurements of viscoelasticity (oscillatory measurements), first the linear viscoelastic region is determined by subjecting the sample to oscillatory stresses of increasing amplitudes and of constant frequency. The moduli are recorded as a function of the amplitude of the stress or of the amplitude of the strain, in order to determine the limits of the linear viscoelastic region. After having identified the linear viscoelastic region, dynamic measurements are made in the linear viscoelastic zone for a constant strain value lying in the linear viscoelastic region and at variable frequency. The Haake RS150 rheometer can cover a range of frequencies varying from 0.0001 to 100 Hz (i.e. 0.00063 to 628 rad/sec).

The following relationships are derived from the values of the amplitudes of the stress τ₀ and of the strain γ₀ and from the loss angle δ:

$$\vert {G}^{*} \vert = \frac{{τ}_{0}}{{γ}_{0}}$$

Surfactant System

The surfactant system used in the composition of the invention which makes it possible to obtain the appearance of a paracrystalline phase during heating to at least 30° C. preferably comprises at least one water-soluble surfactant and at least one water-insoluble surfactant.

The term “water-soluble surfactant” is understood to mean a surfactant which, at a concentration of 20 g/l in deionized water at a temperature of approximately 25° C., gives a transparent isotropic solution.

Conversely, the term “water-insoluble surfactant” is understood to mean a surfactant which, at a concentration of 20 g/l in deionized water at a temperature of approximately 25° C., gives a cloudy solution, indicating nondissolution of the surfactant in water.

Water-soluble Surfactants

Any water-soluble surfactant may be used. They are preferably foaming surfactants, that is to say surfactants capable of foaming in the presence of water. They are mainly anionic, nonionic or amphoteric derivatives having sufficiently short fatty chains for these products to be thoroughly soluble at ambient temperature in the aqueous solvent medium of the composition. A water-soluble surfactant or a mixture of such surfactants may be used.

Useful water-soluble surfactants include for example:

1. Anionic surfactants

According to a specific embodiment of the invention, the surfactant system used preferably comprises at least one water-soluble anionic surfactant and more particularly at least one water-soluble carboxylic acid or one water-soluble carboxylic acid salt, which salt is obtained from the acid and a base. The carboxylic acids which can be used are fatty acids, comprising a saturated or unsaturated linear or branched alkyl chain, having from 6 to 16 carbon atoms and preferably 10 to 14 carbon atoms. The salts of such fatty acids constitute soaps. The fact that the soap is water-soluble or not depends on the length of the alkyl chain and on the counterion constituting the salt. Use may be made, as salts, of, for example, alkali metal salts, alkaline earth metal salts, ammonium salts, aminoalcohol salts and amino acid salts, and in particular of sodium, potassium, magnesium, triethanolamine, N-methylglucamine, lysine and arginine salts. The bases (also known hereinbelow as saponification agents) which can be used to produce these salts can, for example, be inorganic bases, such as alkali metal hydroxides (sodium hydroxide and potassium hydroxide), alkaline earth metal hydroxides (magnesium hydroxide) or ammonium hydroxide, or organic bases, such as triethanolamine, N-methylglucamine, lysine and arginine. The carboxylic acid can in particular be lauric acid or myristic acid.

Mention may be made, as water-soluble soap, of, for example, potassium salts of C₁₀ to C₁₄ fatty acids and in particular the potassium salt of lauric acid, the potassium salt of myristic acid and their mixtures.

Soap is generally introduced into the composition in the form of a base, on the one hand, and of the fatty acid, on the other hand, the formation of the salt taking place in situ. Thus, when the water-soluble soap is composed of the potassium salt of lauric acid and/or of the potassium salt of myristic acid, the composition can then comprise lauric acid and/or myristic acid with a sufficient amount of potassium hydroxide to form the potassium salts of lauric acid and/or of myristic acid.

Mention may be made, as anionic surfactants other than the above-mentioned carboxylic acids and their salts, which can be used in the composition of the invention as water-soluble surfactant, of, for example, ethoxylated carboxylic acids and their salts; sarcosinates and acylsarconisates and their salts, such as sodium lauroyl sarcosinate; taurates and methyltaurates and their salts; isethionates and acylisethionates, reaction products of fatty acids comprising from 10 to 22 carbon atoms with isethionic acid, and their salts, such as sodium isethionate and sodium cocoyl isethionate; sulphosuccinates and their salts; alkyl sulphates and alkyl ether sulphates and their salts, in particular sodium or triethanolamine lauryl sulphate and sodium or potassium lauryl ether sulphate; monoalkyl and dialkyl esters of phosphoric acid and their salts, such as, for example, sodium mono- and dilauryl phosphate, potassium mono- and dilauryl phosphate, triethanolamine mono- and dilauryl phosphate, sodium mono- and dimyristyl phosphate, potassium mono- and dimyristyl phosphate, diethanolamine mono- and dimyristyl phosphate, or triethanolamine mono- and dimyristyl phosphate; alkanesulphonates and their salts; bile salts, such as cholates, deoxycholates, taurocholates or taurodeoxy-cholates; lipoamino acids and their salts, such as mono- and disodium acylglutamates; or bipolar geminal surfactants, such as described in Surfactant Science series, Vol. 74, published by Krister Homberg, and their mixtures.

2. Amphoteric and zwitterionic surfactants

Amphoteric or zwitterionic surfactants which can be used as water-soluble surfactants include for example, betaines, such as dimethylbetaine, coco-betaine and cocamidopropyl betaine; sulphobetaines, such as cocamidopropyl hydroxysultaine; alkylamphoacetates, such as cocoamphodiacetate; and their mixtures.

3. Nonionic surfactants

Useful nonionic surfactants capable of being used as water-soluble surfactants include for example, polyol ethers comprising fatty chains (8 to 30 carbon atoms), such as oxyethylenated sorbitol or glycerol fatty ethers; polyglycerol ethers and esters; polyoxyethylenated fatty alcohols which are ethers formed of ethylene oxide units and of at least one fatty alcohol chain having from 10 to 22 carbon atoms, the solubility of which depends on the ethylene oxide number and on the length of the fatty chain; for example, for a fatty chain comprising 12 carbon atoms, the ethylene oxide number must be greater than 7, and mention may be made, as examples of polyoxyethylenated fatty alcohols, of lauryl alcohol ethers comprising more than 7 oxyethylene groups; alkyl polyglucosides, the alkyl group for which comprises from 1 to 30 carbon atoms, such as, for example, decyl glucoside, lauryl glucoside, cetostearyl glucoside or cocoyl glucoside; alkyl glucopyranosides and alkyl thioglucopyranosides; alkyl maltosides; alkyl-N-methylglucamides; polyoxyethylenated sorbitan esters which generally comprise from 1 to 100 ethylene glycol units and preferably from 2 to 40 ethylene oxide (OE) units; aminoalcohol esters; and their mixtures.

The amount of water-soluble surfactant(s) in the composition of the invention can range, for example, from 10 to 50% by weight preferably from 15 to 35% by weight, with respect to the total weight of the composition. According to a preferred embodiment of the invention, the composition of the invention comprises at least 15% by weight and preferably at least 20% by weight of water-soluble surfactant(s) with respect to the total weight of the composition.

Water-insoluble Surfactants

The water-insoluble surfactants are believed to contribute in particular the texture (consistency) of the final composition. Furthermore, in the temperature range between approximately 25° C. and 45° C., these surfactants partially associate with the water-soluble surfactants to contribute to the formation of the paracrystalline phase (preferably direct hexagonal phase) which, it is believed, is the source of the stability of the product up to at least 45° C.

Useful water-insoluble surfactants used in the composition according to the invention include water-insoluble carboxylic acids and their salts, which salts are obtained from the acid and a base. These salts constitute water-insoluble soaps. The carboxylic acids comprise a saturated or unsaturated, linear or branched alkyl chain, having from 6 to 30 carbon atoms and preferably 12 to 22 carbon atoms. For the derivatives comprising a single saturated fatty chain, the chain advantageously comprises from 12 to 32 carbon atoms, preferably from 14 to 22 carbon atoms and better still from 16 to 20 carbon atoms. For the derivatives comprising a mono-unsaturated or polyunsaturated or branched fatty chain, the chain advantageously comprises from 16 to 34 carbon atoms and preferably from 18 to 24 carbon atoms.

Mention may in particular be made, as carboxylic acid, of palmitic acid and stearic acid.

Use may be made, as salts, of alkali metal salts, alkaline earth metal salts, the ammonium salts, aminoalcohol salts and amino acid salts, and in particular of the sodium, potassium, magnesium, triethanolamine, N-methylglucamine, lysine and arginine salts. The bases which can be used to produce these salts can, for example, be inorganic bases, such as alkali metal hydroxides (sodium hydroxide and potassium hydroxide), alkaline earth metal hydroxides (magnesium hydroxide) or ammonium hydroxide, or organic bases, such as triethanolamine, N-methylglucamine, lysine and arginine.

Mention may be made, for example, as insoluble soap, of the sodium salt of C₁₂ to C₂₂ fatty acids and the potassium salt of C₁₆ to C₂₂ fatty acids and in particular of the potassium salt of palmitic acid and the potassium salt of stearic acid.

The soap is generally introduced into the composition in the form of the base, on the one hand, and of the fatty acid, on the other hand, for formation of the salt taking place in situ. Thus, when the insoluble soap is composed of the potassium salt of palmitic acid and/or of the potassium salt of stearic acid, the composition can then comprise palmitic acid and/or stearic acid with a sufficient amount of potassium hydroxide to form the potassium salts of palmitic acid and/or of stearic acid.

Other surfactants which can be used in the composition of the invention as insoluble surfactant include for example, insoluble nonionic or anionic surfactants, such as esters of glycerol and of fatty acids comprising from 14 to 30 carbon atoms, such as glyceryl stearate; optionally oxyethylenated sterol and phytosterol derivatives; alkaline salts of cholesterol sulphate and in particular the sodium salt; alkaline salts of cholesterol phosphate and in particular the sodium salt; polyoxyethylenated fatty alcohols comprising an oxyethylene chain having a small number of oxyethylene groups and in particular less than 10 oxyethylene groups; dialkyl phosphates, such as alkaline salts of dicetyl phosphate and in particular the sodium and potassium salts; alkaline salts of dimyristyl phosphate and in particular the sodium and potassium salts; lecithins; sphingomyelins; ceramides and their mixtures.

The amount of water-insoluble surfactant(s) in the composition of the invention can range, for example, from 5 to 50% and preferably from 5 to 35% by weight with respect to the total weight of the composition.

The surfactant system (water-soluble and insoluble surfactants) is preferably present in the composition of the invention in an amount, as active material, which can range, for example, from 20 to 65% by weight and preferably ranges from 30 to 65% by weight and better still from 40 to 60% by weight with respect to the total weight of the composition.

The surfactant system preferably comprises an amount of water-soluble soap(s) of at least 10% by weight with respect to the total weight of the composition and an overall amount of (water-soluble and insoluble) soaps preferably of at least 20% by weight with respect to the total weight of the composition and preferably ranging from 30 to 40% by weight with respect to the total weight of the composition.

Useful waxes which can be used in the composition include for example, waxes of animal origin, such as beeswax, lanolin and its derivatives; vegetable waxes, such as candelilla or camauba wax; mineral waxes, such as microcrystalline waxes, paraffin wax or petrolatum wax; synthetic waxes, such as ditrimethylolpropane tetrastearate; and their mixtures. Use is preferably made, as waxes, of camauba wax, candelilla wax, beeswax, and their mixtures.

The viscosities of the creams obtained (viscosity at 25° C. ranging from 2 to 25 Pa·s) and their spreading properties can advantageously be varied by choosing appropriate waxes, as the waxes indicated above exhibit different melting points (from 50° C. to 90° C.) and different hardnesses (from 1 MPa to 10 MPa (MPa=megapascals, hardness measured with the TA/TX2 texture analyser from Rheo at 25° C.)).

The composition comprises at least 0.01% by weight of one or more waxes with respect to the total weight of the composition. The amount of wax can range, for example, from 0.01 to 20% by weight, preferably from 0.1 to 15% by weight and better still from 0.3 to 10% by weight with respect to the total weight of the composition.

The aqueous medium of the foaming creams of the invention, which are preferably intended for a topical application, is a physiologically acceptable medium, that is to say a medium compatible with the skin, mucous membranes, scalp, eyes and/or hair. It can comprise, in addition to water, one or more solvents selected from the group consisting of lower alcohols comprising from 1 to 6 carbon atoms, such as ethanol; polyols, such as glycerol; glycols, such as butylene glycol, isoprene glycol, propylene glycol or polyethylene glycols, such as PEG-8; sorbitol; sugars, such as glucose, fructose, maltose, lactose or sucrose; and their mixtures. The amount of solvent(s) in the composition of the invention can range from 0.5 to 30% by weight and preferably from 5 to 20% by weight with respect to the total weight of the composition.

It is possible to incorporate, in the composition of the invention, one or more polymers. Preferential concentrations range from 0.05 to 2% by weight with respect to the total weight of the composition.

Useful polymers which can be used in the composition of the invention, include:

polysaccharide biopolymers, such as xanthan gum, guar gum, alginates or modified celluloses;

synthetic polymers, such as polyacrylics, for example Carbopol 980, sold by Goodrich, or acrylate/acrylonitrile copolymers, such as Hypan SS201, sold by Kingston;

norganic compounds, such as smectites or modified or unmodified hectorites, such as the Bentone products sold by Rheox, Laponite products sold by Southern Clay Products or the Veegum HS product sold by R. T. Vanderbilt;

their mixtures.

The compositions of the invention can also comprise adjuvants commonly used in the field of foaming cleaners, such as cationic polymers of the polyquaternium type, which contribute softness and creaminess to the foaming cream. These cationic polymers may preferably be selected from the group consisting of the following polymers:

Polyquaternium 5, such as the product Merquat 5 sold by Calgon;

Polyquaternium 6, such as the product Salcare SC 30 sold by Ciba and the product Merquat 100 sold by Calgon;

Polyquaternium 7, such as the products Merquat S, Merquat 2200 and Merquat 550 sold by Calgon and the product Salcare SC 10 sold by Ciba;

Polyquaternium 10, such as the product Polymer JR400 sold by Amerchol;

Polyquaternium 11, such as the products Gafquat 755, Gafquat 755N and Gafquat 734 sold by ISP;

Polyquaternium 15, such as the product Rohagit KF 270 F sold by Rohm;

Polyquaternium 16, such as the products Luviquat FC905, Luviquat FC370, Luviquat HM552 and Luviquat FC550 sold by BASF;

Polyquaternium 22, such as the product Merquat 280 sold by Calgon;

Polyquaternium 28, such as the product Styleze CC10 sold by ISP;

Polyquaternium 39, such as the product Merquat Plus 3330 sold by Calgon;

Polyquaternium 44, such as the product Luviquat Care sold by BASF;

Polyquaternium 46, such as the product Luviquat Hold sold by BASF;

Polyquaternium 47, such as the product Merquat 2001 sold by Calgon.

Mixtures thereof.

Use may also be made, as cationic polymer, of cationic guars, such as the product Jaguar sold by Rhodia.

In addition, the compositions of the invention can comprise one or more adjuvants commonly used in the cosmetic field selected from the group consisting of cosmetic or dermatological active principles, fragrances, preservatives, sequestering agents (EDTA), pigments, pearlescent agents, inorganic or organic fillers, such as talc, kaolin, silica powder or polyethylene powder, soluble dyes, sunscreen agents and their mixtures. The amounts of these various adjuvants are those conventionally used in the field under consideration, for example from 0.01 to 20% of the total weight of the composition. These adjuvants and their concentrations should be such that they do not modify the property desired for the composition of the invention.

Preferred active principles include anti-seborrheic and anti-microbial active principles which make it possible in particular to treat greasy skin. This active principle can be chosen in particular from: β-lactam derivatives, quinolone derivatives, ciprofloxacin, norfloxacin, tetracycline and its salts, erythromycin and its salts, amikacin and its salts, 2,4,4′-trichloro-2′-hydroxydiphenyl ether (or triclosan), 3,4,4′-trichlorocarbanilide (triclocarban), phenoxyethanol, phenoxypropanol, phenoxyisopropanol, doxycycline and its salts, capreomycin and its salts, chlorhexidine and its salts, chlorotetracyline and its salts, oxytetracycline and its salts, clindamycin and its salts, ethambutol and its salts, hexamidine isethionate, metronidazole and its salts, pentamidine and its salts, gentamycin and its salts, kanamycin and its salts, lineomycin and its salts, methacycline and its salts, methenamine and its salts, minocycline and its salts, neomycin and its salts, netilmicin and its salts, paromomycin and its salts, streptomycin and its salts, tobramycin and its salts, miconazole and its salts, amantadine salts, para-chloro-meta-xylenol, nystatin, tolnaftate, salicylic acid and its salts, 5-(n-octanoyl)salicylic acid and its salts, benzoyl peroxide, 3-hydroxybenzoic acid, 4-hydroxybenzoic acid, acetylsalicylic acid, 2-hydroxybutanoic acid, 2-hydroxypentanoic acid, 2-hydroxyhexanoic acid, phytic acid, N-acetyl-L-cysteine acid, lipoic acid, azelaic acid, arachidonic acid, ibuprofen, naproxen, hydrocortisone, acetaminophen, resorcinol, 2,4,4′-trichloro-2′-hydroxydiphenyl ether, 3,4,4′-trichlorocarbanilide, octopirox, lidocaine hydrochloride, clotrimazole, oxtoxyglycerol, octanoylglycine, caprylylglycol, 10-hydroxy-2-decanoic acid, zinc salts, such as zinc gluconate, niacinamide or vitamin B3 (or vitamin PP), and their mixtures.

Use may also be made, in the composition of the invention, as active principles, of any active principle commonly used in the cosmetic and dermatological fields, such as, for example, water-soluble or fat-soluble vitamins or provitamins, such as vitamin A (retinol), vitamin C (ascorbic acid), vitamin B3 or PP (niacinamide), vitamin B(panthenol), vitamin E (tocopherol), vitamin K1, β-carotene, and the derivatives of these vitamins and in particular their esters; steroids, such as DHEA and 7α-hydroxy-DHEA; moisturizing agents, such as glycerol, hyaluronic acid, pyrrolidonecarboxylic acid (PCA) and its salts, sodium pidolate, serine, xylitol, trehalose, ectoin, ceramides or urea; keratolytic and anti-ageing agents, such as α-hydroxy acids, such as glycolic acid, citric acid or lactic acid, or β-hydroxy acids, such as salicylic acid and its derivatives; enzymes and coenzymes and in particular coenzyme Q10; sunscreen agents; optical brighteners; slimming active principles, such as caffeine, theophylline or theobromine; anti-inflammatories, such as 18-β-glycyrrhetinic acid and ursolic acid, and their mixtures.

Use may be made of a mixture of two or more of these active principles. The active principle or principles can, for example, be present in a concentration ranging from 0.01 to 20%, preferably from 0.1 to 10% and better still from 0.5 to 5% of the total weight of the composition.

The compositions according to the invention can constitute in particular foaming creams for topical application used in particular in the cosmetic or dermatological fields as products for cleaning or removing make-up from the skin (body or face, including eyes), scalp and/or hair. They can constitute more particularly a composition for cleansing the skin.

Another subject-matter of the invention is a cosmetic use of the composition as defined above as products for cleaning and/or removing makeup from the skin, scalp and/or hair.

Another subject-matter of the invention is a cosmetic process for cleaning the skin, scalp and/or hair, wherein the composition of the invention is applied to the skin, to the scalp and/or to the hair in the presence of water and in that the foam foamed and the grime are removed by rinsing with water.

The composition according to the invention can advantageously be prepared by using, for at least one stage of the preparation process, a mixing device, such as a roll mill comprising two rolls rotating in opposite directions, between which the paste passes, or a screw mixer-extruder. Preferably, a screw mixer-extruder is used. This process proved to be advantageous in particular when the composition comprises a high level of wax and in particular more than 2% of wax.

Another subject-matter of the invention is thus a process for the preparation of a composition according to the invention, wherein at least one stage of the process is carried out using a screw mixer-exturder.

According to a first embodiment of the invention, the preparation process comprises the following stages:

(1) preparation of a premix of the waxes and other solid compounds at ambient temperature (approximately 25° C.), such as in particular the fatty acids intended to constitute the soaps with the saponification agent, by heating this premix to a temperature at which it melts (approximately to 80° C.),

(2) incorporation of the aqueous phase, also preheated to 80° C., in the premix of stage (1) and mixing of the two phases;

(3) introduction of the mixture obtained into the part, heated to 80° C., of a screw mixer-extruder, this mixer-extruder being subjected to a temperature gradient ranging from 80° C. to 10° C.;

(4) mixing the mixture in the mixer-extruder while cooling it to 10° C. (temperature of the final component of the extruder) as it is conveyed to the outlet of the mixer-extruder; and

(5) incorporation of the saponification agent (base) in the emulsion, in a part of the mixer-extruder where the temperature is close to ambient temperature (i.e., approximately 25° C.).

The components of the screw mixer-extruder which is used are, ranging from the first to the sixth component, brought respectively to the following temperatures: 80° C., 80° C., 80° C., 30° C., 10° C. and 10° C.

The use of the mixer-extruder thus makes it possible to incorporate much higher levels of waxes (2%, 5%, 10%) than conventional mixers of rotor-stator type and to reproducibly obtain a cream of very regular quality and with a viscosity ranging from 20 to 250 poises (2 to 25 Pa·s).

The examples which follow serve to illustrate the invention without, however, exhibiting a limiting nature. The amounts shown are in % by weight, unless otherwise mentioned.

TABLE I

| | Example 1 | Example 2 | Example 3 | Example 4 |
|---|---|---|---|---|
| | according to | according to | according to | according to |
| | the invention | the invention | the invention | the invention |
| Lauric acid | 3 | 3 | 3 | 3 |
| Myristic acid | 20 | 20 | 20 | 20 |
| Palmitic acid | 3 | 3 | 3 | 3 |
| Stearic acid | 3 | 3 | 3 | 3 |
| Glyceryl stearate | 5 | 5 | 5 | 5 |
| (CTFA name: | | | | |
| Glyceryl stearate | | | | |
| SE) | | | | |
| Carnauba wax | 1 | / | / | / |
| Candelilla wax | / | / | 2 | 5 |
| Beeswax | / | 2 | / | / |
| Cocoglucoside (50% | 1% as A.M. | / | / | / |
| as active material | | | | |
| Glycerol | 7 | 7 | 7 | 7 |
| PEG 8 | 7 | 7 | 7 | 7 |
| Preservatives | 0.7 | 0.7 | 0.7 | 0.7 |
| Sequestering agent | 0.2 | 0.2 | 0.2 | 0.2 |
| Potassium hydroxide | 7 | 7 | 7 | 7 |
| Water | q.s. for 100 | q.s for 100 | q.s for 100 | q.s for 100 |
| Appearance | White product | Pearlescent | Pearlescent | Pearlescent |
| | with yellow | white product | white product | white product |
| | pearlescent | | | |
| | highlights | | | |
| pH at 24 h | 9.2 | 9.2 | 9.3 | 9.3 |
| Stability 2 months | Conforms | Conforms | Conforms | Conforms |
| all temperatures | Sharp edges, no | Sharp edges, | Sharp edges, | Sharp edges, |
| | separation, no | no separation, | no separation, | no separation, |
| | change in col- | no change in | no change in | no change in |
| | our or in smell | colour or in | colour or in | colour or in |
| | | smell | smell | smell |
| Viscosity | 212 poises | 236 poises | 165 poises | 236 poises |
| | (21.2 Pa · s) | (23.6 Pa · s) | (16.5 Pa · s) | (23.6 Pa · s) |

The compositions of the invention exhibit the advantage of having a viscosity which is sufficient to constitute a cream which does not flow and which can be packaged in pots, such as a care cream, while retaining good stability and while having a good foaming performance, as is demonstrated in Tables 2 and 3 shown below.

Viscosity characteristics of the compositions indicated above:

TABLE 2

| | | Sweeping | | |
|---|---|---|---|---|
| Manufacturing | Oscillation | \|G*\| at 1 Hz | G′ at 1 Hz | G″ at 1 Hz |
| type | Formula | (Pa) | (Pa) | (Pa) |
| Conventional | Example 1 | 137 700 | 127 800 | 51 220 |
| mixer | according to | | | |
| | the invention | | | |
| Mixer-extruder | Example 1 | 48 970 | 43 680 | 22 140 |
| | according to | | | |
| | the invention | | | |
| | Example 3 | 54 980 | 48 840 | 25 260 |
| | according to | | | |
| | the invention | | | |
| | Example 4 | 113 800 | 110 400 | 27 780 |
| | according to | | | |
| | the invention | | | |

These results show;

that the addition of wax at different levels to the foaming compositions leads to an increase in the viscosity (with a conventional mixer and with an extruder).

and that manufacturing operations with the extruder make it possible to introduce a high level of waxes with acceptable Theological behaviour.

Sensory performance: the foam qualities developed are evaluated according to protocol described below.

Before any use of the products, hands are washed with household soap and then suitably rinsed and dried. The protocol followed is then as follows:

1—the hands are wetted by passing them under running water and shaking them three times to superficially dry them,

2—1 g of product is placed in the hollow of one of the hands,

3—the product is worked between the two palms for 10 seconds,

4—2 ml of water are added and the product is again worked for 10 seconds,

5—the hands are rinsed under water,

6—they are wiped dry.

The criteria are evaluated at each stage of the protocol followed and they are graded on a scale from 0 to 10.

stage 4: evaluation of the foam quality

The foam volume: the grade assigned increases as the volume increases.

The size of the bubbles composing the foam: the grade assigned increases as the bubbles become bigger.

The density: consistency, behaviour of the foam; the grade assigned increases as a density increases.

The softness of the foam: the grade assigned increases as the foam becomes softer.

stage 5: evaluation during rinsing

The rinsing: the grade assigned decreases as a presence of a slippery film which is difficult to remove increases.

The sensory results of each of the criteria are presented in the following Table 3:

TABLE 3

| Criteria | Example according to the invention |
|---|---|
| Foam volume | 5.9 ± 1.0 |
| Bubble size | 3 ± 1.4 |
| Foam density | 7.6 ± 0.8 |
| Foam softness | 8.7 ± 0.8 |
| Rinsing | 8.4 ± 1.1 |

These results show that the composition according to the invention, while having a satisfactory viscosity and a satisfactory stability, also has good foam qualities and in particular that it makes it possible to obtain a fine foam (small size of bubbles) with a good density which is very soft and easy to rinse off.

All articles, references, documents, applications, texts, patents, standards and written materials referred to above are hereby incorporated herein by reference. Where ranges are noted herein, endpoints are included as are all values and subranges between any stated endpoints as if specifically written out.

French patent application 0112151 filed Sep. 20, 2001, is incorporated herein by reference, and priority thereto is hereby claimed.

1. A foaming composition for topical application comprising:
water,
at least one wax, and
a surfactant system,
wherein said composition exhibits a paracrystalline phase selected from the group consisting of direct hexagonal and cubic at a temperature above 30° C. and below 45° C., and wherein said paracrystalline phase remains present up to at least 45° C.

2. The composition according to claim 1, wherein the composition exhibits at least one direct hexagonal phase.

3. The composition according to claim 1, wherein said composition has a complex modulus |G^{*}|, a storage modulus G′ and a loss modulus G″ all having values ranging from 10² to 10⁶ Pa at a temperature of 25° C.

4. The composition according to claim 1, wherein the surfactant system comprises at least one water-soluble surfactant and at least one water-insoluble surfactant.

5. The composition according to claim 1, wherein the surfactant system comprises at least one water-soluble anionic surfactant.

6. The composition according to claim 5, wherein the water-soluble anionic surfactant is selected from the group consisting of carboxylic acids and their salts, ethoxylated carboxylic acids and their salts, sarcosinates and acylsarcosinates and their salts, taurates and methyltaurates and their salts, isethionates and acylisethionates and their salts, sulphosuccinates and their salts, alkyl sulphates and alkyl ether sulphates and their salts, monoalkyl and dialkyl esters of phosphoric acid and their salts, alkanesulphonates and their salts, bile salts, lipoamino acids and their salts, geminal surfactants and their mixtures.

7. The composition according to claim 4, wherein the water-soluble surfactant is an amphoteric or zwitterionic surfactant selected from the group consisting of betaines, sulphobetaines, alkylamphoacetates and their mixtures.

8. The composition according to claim 4, wherein the water-soluble surfactant is a nonionic surfactant selected from the group consisting of polyol ethers, polyglycerol ethers and esters, polyoxyethylenated fatty alcohols, alkyl polyglucosides, alkyl glucopyranosides and alkyl thioglucopyranosides, alkyl maltosides, alkyl-N-methylglucamides, polyoxyethylenated sorbitan esters, aminoalcohol esters and their mixtures.

9. The composition according to claim 4, wherein the water-insoluble surfactant is selected from the group consisting of carboxylic acids and their salts; esters of glycerol and of fatty acids; optionally oxyethylenated sterol and phytosterol derivatives; alkaline salts of cholesterol sulphate; alkaline salts of cholesterol phosphate; polyoxyethylenated fatty alcohols; dialkyl phosphates; lecithins; sphingomyelins; ceramides; and their mixtures.

10. The composition according to claim 1, wherein the surfactant system is present in an amount ranging from 20 to 65% by weight with respect to the total weight of the composition.

11. The composition according to claim 4, comprising from 10 to 50% by weight of water-soluble surfactant(s) with respect to the total weight of the composition.

12. The composition according to claim 4, comprising at least 15% by weight of water-soluble surfactant(s) with respect to the total weight of the composition.

13. The composition according to claim 1, wherein the surfactant system comprises at least 10% by weight of water-soluble soap(s) with respect to the total weight of the composition.

14. The composition according to claim 4, comprising from 5 to 50% by weight of water-insoluble surfactant(s) with respect to the total weight of the composition.

15. The composition according to claim 1, wherein the surfactant system comprises an overall amount of soaps of at least 20% by weight with respect to the total weight of the composition.

16. The composition according to claim 1, wherein the wax is selected from the group consisting of waxes of animal origin; vegetable waxes; mineral waxes; synthetic waxes; and their mixtures.

17. The composition according to claim 1, wherein it comprises at least 0.01% by weight of wax with respect to the total weight of the composition.

18. The composition according to claim 1, wherein it comprises from 0.01 to 20% by weight of wax with respect to the total weight of the composition.

19. The composition according to claim 1, further comprising at least one solvent selected from the group consisting of lower alcohols; polyols; sugars and their mixtures.

20. The composition according to claim 1, further comprising at least one cationic polymer.

21. The composition according to claim 1, further comprising one or more adjuvants selected from the group consisting of cosmetic active principles, fragrances, preservatives, sequestering agents, pigments, pearlescent agents, inorganic or organic fillers, soluble dyes, sunscreen agents and their mixtures.

22. The composition according to claim 1, further comprising an active principle selected from the group consisting of β-lactams, quinolones, ciprofloxacin, norfloxacin, tetracycline and its salts, erythromycin and its salts, amikacin and its salts, triclosan, triclocarban, phenoxyethanol, phenoxypropanol, phenoxyisopropanol, doxycycline and its salts, capreomycin and its salts, chlorhexidine and its salts, chlorotetracyline and its salts, oxytetracycline and its salts, clindamycin and its salts, ethambutol and its salts, hexamidine isethionate, metronidazole and its salts, pentamidine and its salts, gentamycin and its salts, kanamycin and its salts, lineomycin and its salts, methacycline and its salts, methenamine and its salts, minocycline and its salts, neomycin and its salts, netilmicin and its salts, paromomycin and its salts, streptomycin and its salts, tobramycin and its salts, miconazole and its salts, amantadine salts, para-chloro-meta-xylenol, nystatin, tolnaftate, salicylic acid and its salts, 5-(n-octanoyl)salicylic acid and its salts, benzoyl peroxide, 3-hydroxybenzoic acid, 4-hydroxybenzoic acid, acetylsalicylic acid, 2-hydroxybutanoic acid, 2-hydroxypentanoic acid, 2-hydroxyhexanoic acid, phytic acid, N-acetyl-L-cysteine acid, lipoic acid, azelaic acid, arachidonic acid, ibuprofen, naproxen, hydrocortisone, acetaminophen, resorcinol, 2,4,4′-trichloro-2′-hydroxydiphenyl ether, 3,4,4′-trichlorocarbanilide, octopirox, lidocaine hydrochloride, clotrimazole, octoxyglycerol, octanoylglycine, caprylylglycol, 10-hydroxy-2-decanoic acid, zinc salts, such as zinc gluconate, niacinamide, and their mixtures.

23. The composition according to claim 1, in the form of a composition for cleaning the skin.

24. A method for cleaning and/or removing make-up from the skin, scalp and/or hair, comprising applying the composition of claim 1 thereto.

25. A method for cleaning the skin, scalp and/or hair, comprising applying the composition according to claim 1 to the skin, to the scalp and/or to the hair in the presence of water, and removing a foam formed and grime by rinsing with water.

26. A method for cleansing the skin, comprising applying the composition according to claim 1 to the face and/or to the body, and then massaging said composition in the presence of water to develop foam directly on the face and/or body.

27. A method for cleansing, comprising applying the composition according to claim 1 to the palms of the hands followed by developing a foam on the hands by massaging said composition in the presence of water before applying said foam to the face or the body.

28. A process for the preparation of a composition according to claim 1, comprising carrying out at least one stage of the process with a screw mixer-extruder.

29. The process according to claim 26, comprising:
(1) preparation of a premix of the waxe(s) and any other compounds which are solid at ambient temperature by heating this premix at a temperature at which it melts,
(2) incorporation of the aqueous phase, preheated to 80° C., in the premix from stage (1) and mixing of the two phases;
(3) introduction of the mixture obtained in (2) into a part, heated to 80° C., of a screw mixer-extruder, this mixer-extruder being subjected to a temperature gradient ranging from 80° C. to 10° C.;
(4) mixing the mixture in the mixer-extruder in (3) while cooling it to 10° C. as it is conveyed to the outlet of the mixer-extruder; and
(5) incorporation of a saponification agent in the resulting emulsion, in a part of the mixer-extruder where the temperature is at approximately 20°-2 5° C.
